(19) United States
(12) Patent Application Publication
(10) Pub. No.: US 2013/0172648 A1
(43) Pub. Date: Jul. 4, 2013

(54) CATALYST FOR THE PREPARATION OF AROMATIC HYDROCARBONS AND USE THEREOF

(71) Applicant: Saudi Basic Industries Corporation, Riyadh (SA)

(72) Inventor: Subhash Chandra Laha, Vadodara (IN)

(73) Assignee: Saudi Basic Industries Corporation, Riyadh (SA)

(21) Appl. No.: 13/718,015

(22) Filed: Dec. 18, 2012

(30) Foreign Application Priority Data
Jan. 4, 2012 (EP) 12000032.8
Mar. 9, 2012 (EP) 12001629.0

(51) Int. Cl. B01J 29/87; C07C 2/76

(52) U.S. Cl. CPC B01J 29/87; C07C 2/76
USPC 585/412; 585/417; 585/419; 502/61

(57) ABSTRACT

The present invention relates to catalyst composition comprising M₁/Ga/zeolite and La/binder, wherein M₁/Ga/zeolite is a zeolite comprising 0.01-2 wt-% palladium and/or platinum (M₁) with respect to the total M₁/Ga/zeolite and 0.2-2 wt-% gallium (Ga) with respect to the total M₁/Ga/zeolite; and La/binder is a binder comprising 0.5-2 wt-% lanthanum (La) with respect to the total La/binder. Furthermore, the present invention relates to a method for preparing the catalyst composition of the present invention and a process for producing aromatic hydrocarbons comprising contacting a feedstream comprising lower alkanes with the catalyst composition of the present invention under conditions suitable for alkane aromatization.

### CROSS REFERENCE TO RELATED APPLICATIONS

This application claims priority to European Patent Application Serial No. 12000032.8, filed Jan. 4, 2012, and European Patent Application Serial No. 12001629.0, filed Mar. 9, 2012, the contents of which is herein incorporated by reference in its entirety.

The present invention relates to catalyst composition comprising M₁/Ga/zeolite and La/binder, wherein M₁/Ga/zeolite is a zeolite comprising 0.01-2 wt-% palladium and/or platinum (M₁) with respect to the total M₁/Ga/zeolite and 0.2-2 wt-% gallium (Ga) with respect to the total M₁/Ga/zeolite; and La/binder is a binder comprising 0.5-2 wt-% lanthanum (La) with respect to the total La/binder. Furthermore, the present invention relates to a method for preparing the catalyst composition of the present invention and a process for producing aromatic hydrocarbons comprising contacting a feedstream comprising lower alkanes with the catalyst composition of the present invention under conditions suitable for alkane aromatization.

It has been previously described that that lower alkanes can be directly converted into a product stream comprising aromatic hydrocarbons using zeolite-based catalyst.

WO 2008/080517 describes a process wherein aromatic hydrocarbons are produced by contacting lower alkanes with a catalyst composition comprising a gallium containing zeolite and lanthanum modified kaolin as a binder. The nominal lanthanum load of the lanthanum modified binders of WO 2008/080517 is described to be 1 wt-%.

CN1296861 discloses a catalyst useful for hydrocarbon aromatization composed of ZSM-5 with a Si/Al mol ratio of 20-70, further comprising Ga and one metal selected from the group consisting of La, Ag, Pd, Zn and Re. The composition comprises 46-99.4 wt-% ZSM-5; 0.5-2 wt-% Ga; 0.01-2 wt-% of the metal selected from the group consisting of La, Ag, Pd, Zn and Re; and optionally up to 50 wt-% alumina. In a preferred embodiment, the composition comprises 63-99 wt-% ZSM-5; 0.8-1.6 wt-% Ga; 0.1-1 wt-% of the metal selected from the group consisting of La, Ag, Pd, Zn and Re; and optionally up to 35 wt-% alumina.

EP 0 283 212 A1 and U.S. Pat. No. 7,164,052 disclose a process for producing aromatic hydrocarbon compounds comprising contacting C2-C6 hydrocarbons with a catalyst composition comprising gallium and at least one lanthanide element, preferably lanthanum, and a zeolite, preferably MFI/ZSM-5. The zeolite catalyst of EP 0 283 212 A1 may contain from 0.2 to 1 wt-% of gallium and from 0.1 to 2, preferably 0.1 to 0.8 wt-% of rare earth, preferably lanthanum. The zeolite catalyst of U.S. Pat. No. 7,164,052 may contain 0.05 to 10 wt-% gallium and 0.01 to 10 wt-% lanthanide element and based on the total weight of the catalyst composition.

A drawback of conventional zeolite-based catalyst useful in the aromatization of lower alkanes is that the selectivity for aromatics is relatively low. Furthermore, it was found that catalyst activity of conventional zeolite-based catalyst in alkane aromatization process is reduced over time.

It was an object of the present invention to provide a catalyst useful for the aromatization of lower alkanes, having an improved selectivity for useful aromatic hydrocarbons, such as BTX, and which has a more stable catalyst activity.

The solution to the above problem is achieved by providing the embodiments as described herein below and as characterized in the claims. Accordingly, the present invention provides a catalyst composition comprising M₁/Ga/zeolite and La/binder, wherein M₁/Ga/zeolite is a zeolite comprising 0.01-2 wt-% palladium and/or platinum (M₁) with respect to the total M₁/Ga/zeolite and 0.2-2 wt-% gallium (Ga) with respect to the total M₁/Ga/zeolite; and La/binder is a binder comprising 0.5-2 wt-% lanthanum (La) with respect to the total La/binder.

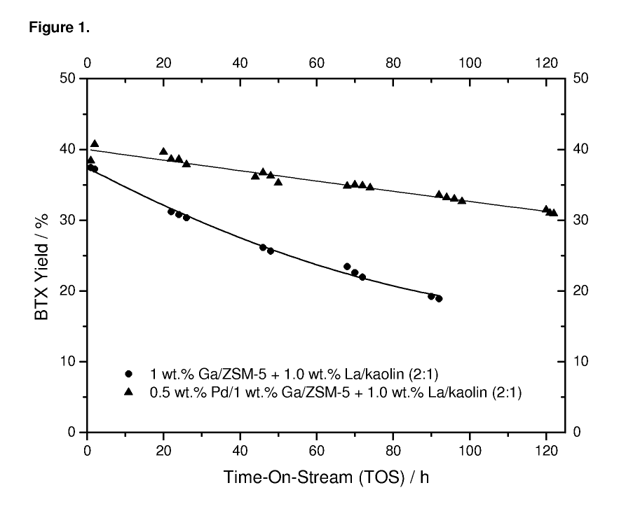

FIG. 1 is a graphical representation of time-on-stream versus BTX yield for two catalyst compositions.

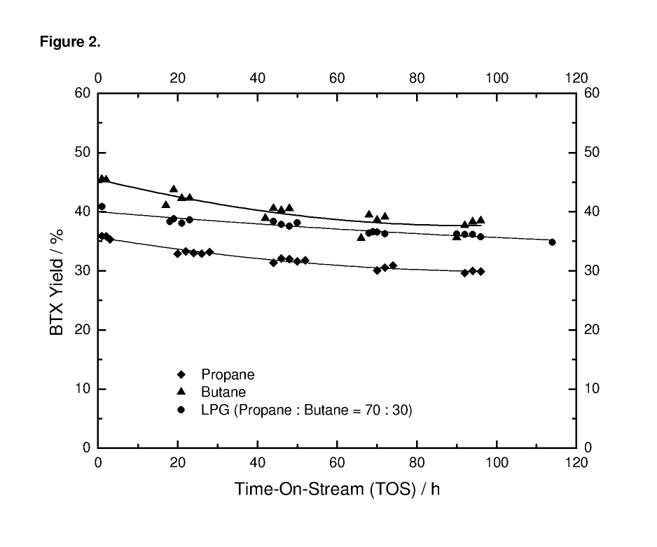

FIG. 2 is a graphical representation of time-on-stream versus BTX yield for propane, butane, and LPG.

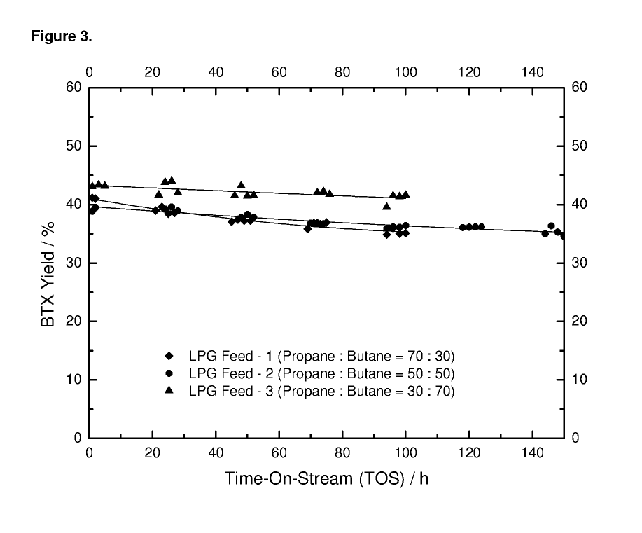

FIG. 3 is a graphical representation of time-on-stream versus BTX yield for LPGs having different propane to butane ratios.

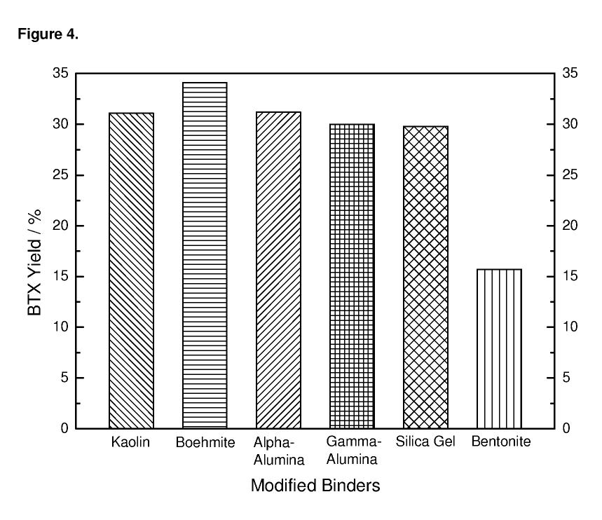

FIG. 4 is a graphical representation of modified binders versus BTX yield.

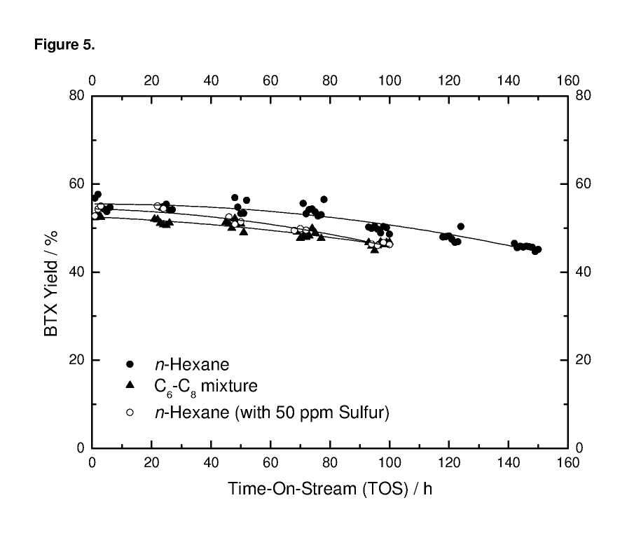

FIG. 5 is a graphical representation of time-on-stream versus BTX yield for n-hexane, C₆-C₈ mixture, and n-hexane with 50 ppm sulfur.

In the context of the present invention, it was found that in an alkane aromatization process, a high alkane conversion rate of 50-70 mol-% and high selectivity for BTX of 50-65 mol-% can be achieved when using the catalyst composition comprising M₁/Ga/zeolite catalyst of the present invention, wherein the M₁/Ga/zeolite catalyst component comprises 0.01-2 wt-% palladium and/or platinum (M₁), and wherein the La/binder comprises 0.5-2 wt-% La. Moreover, it was found that the stability of the catalyst against deactivation is remarkably improved to continuous runs of up to 100-150 hours when compared to conventional bound Pd-comprising zeolite catalysts. These findings are surprising in view of the teachings of the prior art. For instance Shen et al. report in Chinese Chemical Letters 18 (2007) 479-482 that in a propane/methane aromatization process the propane conversion is dramatically decreased when a Ga-modified ZSM-5 catalyst is further modified with Pd, but it promoted the methane conversion.

Accordingly, the catalyst composition provided by the present invention comprises 0.01-2 wt-% palladium and/or platinum with respect to the total M₁/Ga/zeolite, wherein the element selected from the group consisting of palladium and platinum is depicted herein as M₁. Preferably, the catalyst composition comprises M₁/Ga/zeolite comprising 0.02-1.5 wt-% M₁ with respect to the total M₁/Ga/zeolite. More preferably, the catalyst composition comprises M₁/Ga/zeolite comprising 0.03-0.75 wt-% M₁ with respect to the total M₁/Ga/zeolite. Most preferably, the catalyst composition comprises M₁/Ga/zeolite comprising 0.05-0.5 wt-% M₁ with respect to the total M₁/Ga/zeolite.

Furthermore, the catalyst composition comprises M₁/Ga/zeolite comprising 0.2-2 wt-% Ga with respect to the total M₁/Ga/zeolite. Most preferably, the catalyst composition comprises M₁/La/Ga/zeolite comprising 0.5-1.5 wt-% Ga with respect to the total M₁/Ga/zeolite. Selecting the preferred Ga content further improves conversion and BTX selectivity.

The catalyst composition of the present invention optionally comprises zeolite that has been modified with lanthanum (La). This is depicted herein as M₁/La/Ga/zeolite and La/binder, wherein M₁/Ga/zeolite is a zeolite comprising up to 0.5 wt-% lanthanum (La) with respect to the total M₁/Ga/zeolite or as “M₁/La/Ga/zeolite”. Preferably, the catalyst composition comprises M₁/La/Ga/zeolite comprising 0.01-0.1 wt-% lanthanum (La) with respect to the total M₁/La/Ga/zeolite. More preferably, the catalyst composition comprises M₁/La/Ga/zeolite comprising 0.02-0.09 wt-% La with respect to the total M₁/La/Ga/zeolite. Particularly preferably, the catalyst composition comprises M₁/La/Ga/zeolite comprising 0.03-0.08 wt-% La with respect to the total M₁/La/Ga/zeolite. Most preferably, the catalyst composition comprises M₁/La/Ga/zeolite comprising 0.04-0.07 wt-% La with respect to the total M₁/La/Ga/zeolite.

The catalyst composition comprises zeolite. As used herein, the term “zeolite” or “aluminosilicate zeolite” relates to an aluminosilicate molecular sieve. These inorganic porous materials are well known to the skilled person. An overview of their characteristics is for example provided by the chapter on Molecular Sieves in Kirk-Othmer Encyclopedia of Chemical Technology, Volume 16, p 811-853; in Atlas of Zeolite Framework Types, 5^{th} edition, (Elsevier, 2001). Preferably, the zeolite is a medium pore size aluminosilicate zeolite. Most preferably the zeolite is ZSM-5 zeolite, which is a well-known zeolite having MFI structure. Other suitable zeolites include, but are not limited to, MCM-22 and ZSM-11. The term “medium pore zeolite” is commonly used in the field of zeolite catalysts. Accordingly, a medium pore size zeolite is a zeolite having a pore size of 5-6 Å. Suitable medium pore size zeolites are 10-ring zeolites. i.e. the pore is formed by a ring consisting of 10 SiO₄ tetrahedra. Zeolites of the 8-ring structure type are called small pore size zeolites; and those of the 12-ring structure type, like for example beta zeolite, are also referred to as large pore sized. In the above cited Atlas of Zeolite Framework Types various zeolites are listed based on ring structure.

The zeolite of the present invention may be dealuminated. Preferably, the silica (SiO₂) to alumina (Al₂O₃) molar ratio of the ZSM-5 zeolite is in the range of 10-200. Means and methods to obtain dealuminated zeolite are well known in the art and include, but are not limited to the acid leaching technique; see e.g. Post-synthesis Modification I; Molecular Sieves, Volume 3; Eds. H. G. Karge, J. Weitkamp; Year (2002); Pages 204-255. In the context of the present invention it was found that using a dealuminated zeolite having a SiO₂ to Al₂O₃ molar ratio of 10-200 improves the performance/stability of the catalyst. Means and methods for quantifying the SiO₂ to Al₂O₃ molar ratio of a dealuminated zeolite are well known in the art and include, but are not limited to AAS (Atomic Absorption Spectrometer) or ICP (Inductively Coupled Plasma Spectrometry) analysis.

It is preferred that the zeolite is in the hydrogen form: i.e. having at least a portion of the original cations associated therewith replaced by hydrogen. Methods to convert an aluminosilicate zeolite to the hydrogen form are well known in the art. A first method involves direct ion exchange employing an acid. A second method involves base-exchange using ammonium salts followed by calcination.

The catalyst composition of the present invention comprises a binder that is modified with La (La/binder). Any conventional catalyst binder that can be modified with La may be used. It is well within the scope of the skilled person to select a suitable binder; see Otterstedt et al (1998). Preferably, the binder is selected from the group consisting of kaolin, boehmite, alumina, and silica. More preferably, the binder is kaolin or boehmite. The alumina binder may be alpha alumina or gamma alumina. The catalyst composition of the present invention preferably comprises 5-50 wt-% La/binder with respect to the total catalyst composition.

In a further aspect of the present invention a method for preparing a catalyst composition is provided. Accordingly, the present invention provides a method for preparing the catalyst composition as described herein comprising the steps of:

(i) depositing Ga on zeolite to provide Ga/zeolite;

(ii) depositing M₁ on Ga/zeolite to provide M₁/Ga/zeolite;

(iii) depositing La on the binder to provide La/binder; and

(iv) combining said M₁/Ga/zeolite and said La/binder.

In one embodiment, the method for preparing a catalyst composition according to the present invention further comprises the step of depositing La on the zeolite before depositing M₁. Accordingly, when the catalyst composition to be prepared comprises a zeolite that is modified with La, the method comprises a process step wherein La is deposited on Ga/zeolite. In this case, the present invention provides a method for preparing the catalyst composition comprising the steps of

depositing Ga on zeolite to provide Ga/zeolite;

depositing La on Ga/zeolite to provide La/Ga/zeolite;

depositing M₁ on La/Ga/zeolite to provide M₁/La/Ga/zeolite;

depositing La on the binder to provide La/binder; and

combining said M₁/La/Ga/zeolite and said La/binder.

Optionally, the Ga and La are deposited on the zeolite simultaneously, e.g. by ion-exchange and/or impregnation with a solution comprising a soluble salt of gallium (Ga) and lanthanum (La). In that case, the present invention provides a method for preparing the catalyst composition comprising the steps of

depositing La and Ga on zeolite to provide La/Ga/zeolite;

depositing M₁ on La/Ga/zeolite to provide M₁/La/Ga/zeolite;

depositing La on the binder to provide La/binder; and

combining said M₁/La/Ga/zeolite and said La/binder.

Preferably, the Ga is deposited by ion-exchange and/or impregnation with a solution comprising a soluble salt of gallium (Ga). Preferably, the Ga-salt solution is an aqueous solution. A preferred Ga salt used to prepare the solution is gallium(III) nitrate.

Preferably, the La is deposited by ion-exchange and/or impregnation with a solution comprising a soluble salt of lanthanum (La). Preferably, the La-salt solution is an aqueous solution. A preferred La salt used to prepare the solution is lanthanum(III) nitrate. Preferably, the solution comprising lanthanum (La) salt used to deposit La on the zeolite comprises 0.001-0.01 M La, more preferably 0.002-0.006 M La. The solution comprising lanthanum (La) salt used to deposit La on the binder preferably comprises 0.01-0.1 M La, more preferably 0.02-0.06 M La. The solution comprising lanthanum (La) salt used to deposit La on the binder generally comprises a higher concentration of La than the solution comprising La salt used to deposit La on the zeolite.

Preferably, M₁/Ga/zeolite is prepared in the above defined M₁/Ga/zeolite preparation step (ii) by ion-exchange and/or impregnation with a solution comprising a soluble salt of palladium and/or platinum (M₁). Preferably, the M₁-salt solution is an aqueous solution. Preferred M₁ salts used to prepare the solution are selected from the group consisting of tetraamine metal chlorine salts, wherein the metal is Pd or Pt.

For incipient wetness or wetness impregnation, as used in the present invention, a minimum amount of solvent, preferably water, is used to dissolve the metal salt which as aqueous solution of the salt is just sufficient to soak the catalyst or the binder and prepare a dry thick paste. Since the lanthanum loading on the binder may be 10-20 times to that of the concentration on the catalyst, different concentrations of the La-solution are preferred for effective impregnation of lanthanum in the catalyst as well as in the binder.

In a further embodiment of the present invention, a process is provided for producing a product stream comprising aromatic hydrocarbons, wherein the catalyst composition as described herein is contacted with a feedstream comprising lower alkanes.

The lower alkanes that are preferably comprised in the feedstream are C₂-C₈ alkanes (i.e. alkanes having 2-8 carbon atoms), preferably C₃-C₄ alkanes.

Other lower alkanes which are preferably comprised in the feedstream are C₆-C₈ alkanes. It was surprisingly found that also light naphtha, such as C₆ and C₈ alkanes, can be efficiently converted into aromatic hydrocarbons using the process of the present invention. Furthermore, it was surprisingly found that the aromatization process of the present invention is relatively insensitive to sulphur impurities, such as thiophene, in the lower alkane feed.

The terms “aromatic hydrocarbon” is very well known in the art. Accordingly, the term “aromatic hydrocarbon” relates to cyclically conjugated hydrocarbon with a stability (due to delocalization) that is significantly greater than that of a hypothetical localized structure (e.g. Kekulé structure). The most common method for determining aromaticity of a given hydrocarbon is the observation of diatropicity in the ¹H NMR spectrum. Preferably, the aromatic hydrocarbons produced in the process of the present invention are aromatic hydrocarbons having between 6 and 12 carbon atoms (C₆-C₁₂ aromatics). More preferably, the hydrocarbons produced in the process of the present invention are BTX, which is a commonly known abbreviation of a mixture of benzene, toluene and xylenes.

The process of the present invention is performed at conditions suitable for alkane aromatization, also described herein as “alkane aromatization conditions”, which can be easily determined by the person skilled in the art; see e.g. O'Connor, Aromatization of Light Alkanes. Handbook of Heterogeneous Catalysis Wiley-VCH 2008, pages 3123-3133. Accordingly, the process of the present invention is preferably performed at a temperature of 450-600° C. and a weight hourly space velocity (WHSV) of 0.5-5.0.

Furthermore, a process for producing a product stream comprising aromatic hydrocarbons is provided comprising the steps of:

preparing the catalyst composition of the present invention by the method comprising the steps of:

(i) depositing Ga on zeolite to provide Ga/zeolite;

(ii) depositing M₁ on Ga/zeolite to provide M₁/Ga/zeolite,

(iii) depositing La on the binder to provide La/binder, and

(iv) combining said M₁/Ga/zeolite and said La/binder; and

contacting the catalyst composition with a feedstream comprising lower alkanes.

In case the catalyst composition to be prepared comprises a zeolite that is further comprises La, the above method also comprises a process step wherein La is deposited on Ga-zeolite. Optionally, the Ga and La are deposited on the zeolite simultaneously, e.g. by ion-exchange and/or impregnation with a solution comprising a soluble salt of gallium (Ga) and lanthanum (La).

### MODE(S) FOR CARRYING OUT THE INVENTION

The present invention will now be more fully described by the following non-limiting Examples.

### Preparation of Ga/ZSM-5 Zeolite

0.5714 g gallium nitrate was dissolved in 200 ml demineralised water in a 3-neck round bottom flask. 10 g of dry ZSM-5 in NH₄ form, having a Si/Al ratio of 25 was added. The mixture was heated to 90-95° C. and stirred at 300 rpm for 4 hrs. The Ga-exchanged ZSM-5 was filtered, washed with 2 litres of demineralised water and dried in air oven at 120° C. for overnight. The Ga content on the zeolite was determined by AAS and ICP to be around 1 wt %. This procedure can be applied to prepare Ga exchanged ZSM-5 with other Si/Al ratios.

### Preparation of La/Ga/ZSM-5 Zeolite

0.0156 g lanthanum nitrate hexahydrate was dissolved in 10 ml demineralised water. 10 g of dry Ga/ZSM-5 was taken on a Petri dish and lanthanum nitrate solution was added dropwise to the Ga/ZSM-5 and mixed well to make a thick homogenous paste. The paste was dried in air oven at 120° C. for overnight and then calcined at 550° C. in zero air with the flow of 100 ml/min for 4 hrs. The La content on the zeolite was determined by ICP to be around 0.05 wt %. This procedure can be applied to prepare La/Ga/ZSM-5 catalysts with different La-composition.

### Preparation of Pd/La/Ga/ZSM-5 Zeolite

0.0125 g tetraamminepalladium (II) chloride monohydrate was dissolved in 10 ml demineralised water. 10 g of dry La/Ga/ZSM-5 was taken on a Petri dish and tetraamminepalladium (II) chloride solution was added dropwise to the La/Ga/ZSM-5 and mixed well to make a thick homogenous paste. The paste was dried in air oven at 120° C. for overnight and then calcined at 550° C. in zero air with the flow of 100 ml/min for 4 hrs. The Pd content on the zeolite was determined by ICP to be around 0.05 wt %. This procedure can be applied to prepare Pd/La/Ga/ZSM-5 catalysts with different Pd-composition.

### Preparation of Support Materials (Binders)

0.3118 g lanthanum nitrate hexahydrate was dissolved in 15-20 ml demineralised water. 10 g of kaolin was taken on a Petri dish and lanthanum nitrate solution was slowly added to kaolin to make a thick homogenous paste. The paste was dried in air oven at 120° C. for overnight and then calcined at 550° C. in zero air with the flow of 100 ml/min for 4 hrs. The La content on the zeolite was determined by ICP to be around 1.0 wt %. This procedure can be applied to prepare La/kaolin binders with different La-composition. Further, this procedure can be applied to prepare different La/binders.

### Preparation of Catalyst Particles

A number of catalyst compositions comprising different zeolites and binder supports were prepared in particle form by mixing thoroughly the zeolite and the binder support in 2:1 ratio. The mixture was pressed at 10 ton pressure to make pellets. The pressed catalyst compositions were crushed, sieved. The fraction containing particles from 0.25 to 0.5 mm and the fraction containing particles from 0.5 to 1.00 mm particles were selected for further use. The particles of the active zeolite component, and modified La/binder can be prepared separately and then mix the two components (particles) in 2:1 ratio (wt-/wt-) to prepare the final catalyst composition and perform the catalytic testing.

### Catalyst Testing

Two grams catalyst particles (particle size 0.25-0.5 mm) were loaded in a down flow fixed bed micro catalytic reactor and pre-treated in the following way:

Step 1: Exposed for 1 h to moisture-free air flow of 25 ml/min at 600° C.;

Step 2: Exposed for 1 h to 50 ml/min hydrogen flow at 525° C.

After the pre-treatment, the process was switched to the lower alkane feed.

For the propane aromatization process, propane was fed to the bed at a rate of 23.33 ml/min. The temperature of the catalyst bed before start of propane flow was 525° C. The Weight Hourly Space Velocity (WHSV) was 1.4 h⁻¹.

For the n-hexane aromatization process, n-hexane (optionally further comprising 50 ppm wt of thiophene) was fed to the bed, wherein the catalyst bed was kept at a temperature of 500° C. The Weight Hourly Space Velocity (WHSV) was 2.0 h⁻¹.

For the C₆-C₈ alkane aromatization process, a mixture of C₆-C₈ alkanes consisting of 62.5 wt-% C₆ alkane, 27.5 wt-% C₇ alkane and 10 wt-% C₈ alkane was fed to the bed, wherein the catalyst bed was kept at a temperature of 500° C. The Weight Hourly Space Velocity (WHSV) was 2.0 h⁻¹.

Unconverted lower alkane and formed products were analysed by an on-line Gas Chromatograph, separation column Petrocol DH 50.2, using a Flame Ionization Detector.

After the reaction the catalyst was regenerated in the following way:

Step 1: Exposed for 4 h in nitrogen gas (270 ml/min) with 2 vol-% of moisture-free air at 540° C.;

Step 2: The reactor was cooled to 150° C., start passing steam with nitrogen for 30 min (N₂ flow=50 ml/min, Water flow=0.0021 ml/min). This step is optional and was carried out once after five cycles (approx.)

Step 3: Increased the reactor temperature up to 525° C. with nitrogen gas (76 ml/min)

Step 4: Exposed for 30 min to 50 ml/min hydrogen flow at 525° C.

After the regeneration of the catalyst, propane was fed to the bed at a rate of 23.33 ml/min and propane aromatization reaction was continued.

The provided values have been calculated as follows:

### Conversion:

An indication of the activity of the catalyst was determined by the extent of conversion of the propane or for more active catalysts by the extent of volume reduction of the reagent gases (using nitrogen as internal standard). The basic equation used was:

Conversion %=Moles of propaneᵢₙ−moles of propaneₒᵤₜ/moles of propaneᵢₙ*100/1

### Selectivity

First of all, the varying response of the detector to each product component was converted into % v/v by, multiplying them with online calibration factors. Then these were converted into moles by taking account the flow out of internal standard, moles of feed in and time in hours. Moles of each product were converted into mole-% and selectivity-% was measured by taking carbon numbers into account.

### Yield

The yield of given process product was calculated by multiplying the conversion with the fraction of selectivity.

FIG. 1 provides a comparison of BTX yield for propane aromatization and effect of promoter (Pd) in the catalyst (Reaction temperature=525° C., Pressure=1 atmosphere, WHSV=1.4 h⁻¹). Active component to binder ratio is considered as 2:1 (wt/wt) for the final catalysts presented below. It is demonstrated that the catalyst comprising ‘Pd’ in the active component (Ga/ZSM-5) produced higher BTX yield and also showed high resilience against deactivation of the catalyst for a continuous reaction run of more than 100 hours in comparison to the catalyst without ‘Pd’ in the active component.

FIG. 2 provides a comparison of BTX yield for propane, butane and LPG (Propane:Butane=70:30) aromatization and effect of promoter (Pd and La) in the catalyst (Catalyst=0.05 wt. % Pd/0.05 wt. % La/1 wt. % Ga/ZSM-5+1 wt % La/Kaolin (2:1), Reaction temperature=525° C., Pressure=1 atmosphere, WHSV=1.4-1.8 h⁻¹). Active component to binder ratio is considered as 2:1 (wt/wt) for the final catalysts presented below. It is observed that the catalyst produced higher BTX yield and resilience against deactivation for a continuous reaction run of ≧100 hours with the change in feed composition from propane to butane.

FIG. 3 provides a comparison of BTX yield for LPG aromatization with different feed composition (Propane:Butane=70:30, 50:50 and 30:70) (Catalyst=0.05 wt. % Pd/0.05 wt. % La/1 wt. % Ga/ZSM-5+1 wt % La/Kaolin (2:1), Reaction temperature=525° C., Pressure=1 atmosphere, WHSV=1.5-1.7 h⁻¹). Active component to binder ratio is considered as 2:1 (wt/wt) for the final catalysts presented below. Similar observation, as shown in FIG. 2, is recorded while performing catalytic performance study using different LPG feed composition. BTX yield increased for a continuous reaction run of ≧100 hours with the change in the LPG feed composition (propane-rich to butane-rich feed).

FIG. 4 provides a comparison of BTX yield for propane aromatization and effect of binders in the catalyst (Reaction temperature=525° C., Pressure=1 atmosphere, WHSV=1.4 h⁻¹, Reaction time=20 hours). Active component to binder ratio is considered as 2:1 (wt/wt) for the final catalysts presented below. It is demonstrated that the catalyst comprising modified binders (1% La/binders) produced comparable BTX yield except bentonite after a continuous reaction run of 20 hours.

FIG. 5 provides a comparison of BTX yield for light naphtha aromatization and effect of sulfur in the n-hexane feed (Reaction temperature=500° C., Pressure=1 atmosphere, WHSV=2.0 h⁻¹). Active component to binder ratio is considered as 2:1 (wt/wt) for the final catalysts presented below. It is observed that the catalyst produced high BTX yield and resilience against deactivation for a continuous reaction run of ≧100 hours with n-hexane and C₆-C₈ mixture (C₆: 62.5 wt-%, C₇: 27.5 wt-% and C₈: 10 wt-%) as feed. Presence of 50 ppm (weight) of sulfur (thiophene) in the feedstream did not exert any significant adverse effect on the aromatization of n-hexane for a continuous run up to 100 hours.

TABLE 1
Effect of Pd and its concentration on the catalytic performance for propane
aromatization reaction

| | | Propane | | |
|---|---|---|---|---|
| | | Con- | BTX | BTX |
| | Reaction | version/ | Selectivity/ | Yield/ |
| Catalysts | Time/h | % | % | % |
| 1% Ga/ZSM-5 + | 48 | 48.8 | 58.0 | 28.3 |
| 1% La/kaolin (2:1) | | | | |
| (Comparative) | | | | |
| 0.25% Pd/1% Ga/ZSM-5 + | 48 | 43.6 | 51.3 | 22.4 |
| 1% La/kaolin (2:1) | | | | |
| 0.5% Pd/1% Ga/ZSM-5 + | 48 | 60.9 | 59.5 | 36.2 |
| 1% La/kaolin (2:1) | | | | |
| 0.75% Pd/1% Ga/ZSM-5 + | 48 | 55.2 | 56 | 30.9 |
| 1% La/kaolin (2:1) | | | | |
| 1.0% Pd/1% Ga/ZSM-5 + | 48 | 67.1 | 48.4 | 32.5 |
| 1% La/kaolin (2:1) | | | | |

TABLE 2
Comparison of reproducibility studies of catalysts for propane
aromatization reaction with catalyst composition comprising
0.5% Pd/1% Ga-HZSM-5(25) + 1% La/kaolin (2:1) as
principal components

| | Reaction | Propane | BTX | |
|---|---|---|---|---|
| Catalysts | Time/h | Conversion/% | Selectivity/% | BTX Yield/% |
| Batch 1 | 1 | 80.1 | 47.9 | 38.4 |
| Batch 2 | 1 | 72.9 | 49.9 | 36.4 |
| Batch 3 | 1 | 72.0 | 50.1 | 36.1 |
| Batch 4 | 1 | 68.5 | 56.6 | 38.8 |
| Batch 5 | 1 | 75.1 | 55.7 | 41.8 |

TABLE 3
Comparison of catalysts stability studies for propane aromatization
reaction using catalyst composition comprising 0.5% Pd/1%
Ga-HZSM-5(25) + 1% La/kaolin (2:1) as principal components

| No. | Reaction | Propane | BTX | |
|---|---|---|---|---|
| of Cycles | Time/h | Conversion/% | Selectivity/% | BTX Yield/% |
| Cycle 1 | 1 | 80.1 | 47.9 | 38.4 |
| Cycle 2 | 1 | 75.6 | 50.9 | 38.5 |
| Cycle 3 | 1 | 75.4 | 49.6 | 37.4 |
| Cycle 4 | 1 | 75.0 | 55.8 | 41.9 |

TABLE 4
Comparison of reproducibility studies of catalysts for LPG
(Propane:Butane = 70:30) aromatization reaction with catalyst
composition comprising 0.05% Pd/0.05% La/1% Ga-HZSM-5(25) +
1% La/kaolin (2:1) as principal components

| | Reaction | Propane | BTX | |
|---|---|---|---|---|
| Catalysts | Time/h | Conversion/% | Selectivity/% | BTX Yield/% |
| Batch 1 | 1 | 62.9 | 62.6 | 39.4 |
| Batch 2 | 1 | 65.5 | 62.4 | 40.9 |
| Batch 3 | 1 | 65.1 | 62.2 | 40.5 |
| Batch 4 | 1 | 65.2 | 60.9 | 39.7 |

TABLE 5
Comparison of catalysts stability studies for LPG
(Propane:Butane = 70:30) aromatization reaction with catalyst
composition comprising 0.05% Pd/0.05% La/1% Ga-HZSM-5(25) +
1% La/kaolin (2:1) as principal components

| | | Propane | BTX | |
|---|---|---|---|---|
| Catalysts | Time/h | Conversion/% | Selectivity/% | BTX Yield/% |
| Cycle 1 | 1 | 65.5 | 62.4 | 40.9 |
| Cycle 2 | 1 | 64.1 | 64.3 | 41.2 |
| Cycle 3 | 1 | 63.1 | 65.6 | 41.4 |
| Cycle 4 | 1 | 64.9 | 63.1 | 41.0 |

1. A catalyst composition comprising M₁/Ga/zeolite and La/binder,
wherein the M₁/Ga/zeolite is a zeolite comprising 0.01-2 wt-% palladium and/or platinum (M₁) and 0.2-2 wt-% gallium (Ga), with respect to a total weight of the M₁/Ga/zeolite; and
wherein the La/binder is a binder comprising 0.5-2 wt-% lanthanum (La), with respect to a total weight of the La/binder.

2. The catalyst composition according to claim 1, wherein the M₁/Ga/zeolite further comprises up to 0.5 wt-% lanthanum (La), with respect to the total weight of the M₁/La/Ga/zeolite.

3. The catalyst composition according to claim 1, wherein the binder is selected from the group consisting of kaolin, boehmite, alumina, and silica.

4. The catalyst composition according to claim 3, wherein the binder is kaolin or boehmite.

5. The catalyst composition according to claim 1, wherein the catalyst composition comprises 5-50 wt-% La/binder with respect to a total weight of the catalyst composition.

6. The catalyst composition according to claim 1, wherein the zeolite is ZSM-5 zeolite.

7. The catalyst composition according to claim 6, wherein the silica to alumina molar ratio of the ZSM-5 zeolite is in the range of 10-200.

8. A method for preparing a catalyst composition, comprising:
(i) depositing Ga on a zeolite to form a Ga/zeolite;
(ii) depositing M₁ on the Ga/zeolite to form a M₁/Ga/zeolite;
(iii) depositing La on a binder to form a La/binder; and
(iv) combining the M₁/Ga/zeolite and the La/binder to form the catalyst composition;
wherein M₁/Ga/zeolite is a zeolite comprising 0.01-2 wt-% palladium and/or platinum (M₁) and 0.2-2 wt-% gallium (Ga), with respect to a total weight of the M₁/Ga/zeolite; and
wherein the La/binder comprises 0.5-2 wt-% lanthanum (La), with respect to a total weight of the La/binder.

9. The method according to claim 8, wherein La is deposited on the zeolite before depositing M₁.

10. The method according to claim 8, wherein in step (ii) M₁ is deposited by ion-exchange and/or impregnation with a solution comprising a soluble salt of M₁.

11. The method according to claim 8, wherein in step (iii) the La is deposited on the binder by impregnation of said binder with a solution comprising a soluble salt of lanthanum (La).

12. A process for producing a product stream comprising aromatic hydrocarbons, comprising
contacting a catalyst composition with feedstream comprising lower alkanes to produce the product stream comprising aromatic hydrocarbons;
wherein the catalyst composition comprises M₁/Ga/zeolite and La/binder;
wherein the M₁/Ga/zeolite is a zeolite comprising 0.01-2 wt-% palladium and/or platinum (M₁) and 0.2-2 wt-% gallium (Ga), with respect to a total weight of the M₁/Ga/zeolite; and
wherein the La/binder is a binder comprising 0.5-2 wt-% lanthanum (La), with respect to a total weight of the La/binder.

13. The process according to claim 12, wherein the product stream comprises benzene, toluene and xylenes.

14. The process according to claim 12, wherein the lower alkanes are C₃-C₈ alkanes.

15. The process according to claim 12, wherein the process is performed at a temperature of 450-600° C. and a weight hourly space velocity (WHSV) of 0.5-5.0 h⁻¹.
